(19) United States
(12) Patent Application Publication
(10) Pub. No.: US 2014/0107630 A1
(43) Pub. Date: Apr. 17, 2014

(54) SIDE FIRING OPTICAL FIBER DEVICE FOR CONSISTENT, RAPID VAPORIZATION OF TISSUE AND EXTENDED LONGEVITY

(71) Applicant: Trimedyne, Inc., Irvine, CA (US)

(72) Inventors: Glenn D. Yeik, Lake Forest, CA (US); Cristobal R. Hernandez, Santa Ana, CA (US)

(21) Appl. No.: 14/039,081

(22) Filed: Sep. 27, 2013

Related U.S. Application Data
(60) Provisional application No. 61/706,517, filed on Sep. 27, 2012.

(51) Int. Cl. F21V 8/00; A61F 9/008

(52) U.S. Cl. CPC G02B 6/001; A61F 9/008
USPC 606/5; 362/553

(57) ABSTRACT

A side firing laser device suitable for use in medical and surgical procedures has a laser energy transmission efficiency in excess of 90% and provides consistent and rapid vaporization of tissue as well as long useful life. The device includes a conduit with an optical fiber therewithin. The optical fiber is adapted for coupling to a laser energy source at the proximal end thereof and has a beveled distal end portion capped by a closed end capillary tube which, in turn, is surrounded by a reflective sheath with a side port through which laser energy emitted by the optical fiber can pass.

### CROSS-REFERENCE TO RELATED APPLICATION

This application claims the benefit of U.S. Provisional Application No. 61/706,517, filed on Sep. 27, 2012, which is incorporated herein by reference in its entirety.

### FIELD OF INVENTION

This invention relates to improved fiber optic laser energy delivery devices.

### BACKGROUND OF THE INVENTION

Commercially available glass optical fibers efficiently transmit light at various wavelengths. Such glass optical fibers typically have a fused silica core, sometimes also referred to as a quartz, core, surrounded by a cladding of fused silica doped with materials to lower its refractive index to a level lower than that of the core, an optional polymer cladding with a lower refractive index than that of the glass cladding, which increases the effective numerical aperture of the optical fiber, and a buffer coating that protects the optical fiber from mechanical damage.

For efficient transmission of wavelengths of about 190 nm to 1100 nm, the core of the optical fiber must have a high hydroxyl ion content, known as a “High-OH Fiber” (a hydroxyl ion content of about 600 to 1000 ppm), and the glass cladding would typically, but may not, also have a High-OH content.

For efficient transmission of wavelengths from about 500 nm to 2300 nm, some of which wavelengths of light (between 1400 nm to 1500 nm and between 1800 nm to 2300 nm) are highly absorbed by water, the core of the optical fiber must have a low hydroxyl ion content, known as a “Low-OH Fiber” (hydroxyl ion content of about 0.1 to 100 ppm), and the glass cladding would typically, but may not, also have a Low-OH content.

When used in medical procedures, such glass optical fibers typically have a core to cladding ratio of about 1:1.1 to 1:1.2. Drawing custom fibers with either larger core to cladding ratios can entail special production runs, delays in delivery and possibly substantial additional cost.

To allow such an optical fiber to deliver light substantially laterally to the longitudinal axis of the optical fiber, the buffer coating and any polymer cladding may be removed from the distal end portion of the optical fiber, hereinafter referred to “baring” or a “bared” optical fiber, the core and cladding of the optical fiber may be beveled at an angle of about 33° to 45°, and the bared portion of the optical fiber may be disposed within in a distally closed-ended capillary tube. Commonly, the capillary tube is fixed in place over the bared, beveled, distal end portion of the optical fiber by fusing the capillary tube to the bared portion of the optical fiber at a high temperature. Alternatively, the capillary tube may be attached to the optical fiber by the use of an adhesive, a combination of thermal fusing and an adhesive or other means known in the art. The capillary tube creates an air environment opposite the distal, beveled end surface of the optical fiber.

Laser energy or power transmitted through such a side firing optical fiber device, hereinafter referred to as a “side firing fiber” or “side firing device”, is emitted laterally from the axis of the optical fiber by total internal reflection, which requires fluid interface with an index of refraction significantly lower than the index of refraction of the core of the optical fiber, such as air, opposite the beveled distal end surface of the optical fiber. According to Snell's law, within known limitations, laser energy or power will be emitted from such an optical fiber device at an angle double the angle of the beveled, distal end of the optical fiber.

Directing light laterally from the axis of a side firing fiber is useful in many medical applications, including high laser energy or power applications such as vaporizing a portion of the lobes of an enlarged prostate that is obstructing urine flow, a condition called benign prostatic hyperplasia or “BPH”, or vaporizing a portion of the nucleus pulposa of a herniated spinal disc, to relieve the pressure of the disc on nerves in the spine, which causes pain, vaporizing a solid tumor and other applications.

Conventional side firing devices suffer from several light transmission efficiency losses, including but not limited to at least one of the following: (a) many of the higher order light modes impinging on the beveled, distal end surface of the optical fiber at angles higher than those which allow total internal reflection, causing a portion of the transmitted light to be emitted in directions other than the primarily intended direction; (b) aberrant reflections of light arising from the beveled, distal end surface of the optical fiber which, even if very carefully beveled and polished, may not be absolutely flat; (c) aberrant reflections of light arising from the interface between the interior surface of the capillary tube and the outer surface of the bared optical fiber; (d) diminished transparency of the capillary tube due to damage to and erosion of the capillary tube from various sources; (e) aberrant reflections of light arising from the capillary tube acting as a waveguide and transmitting light forward and backward along the longitudinal axis of the optical fiber; (f) aberrant emissions of light from damage to the thin, beveled distal edge or end surface of the optical fiber from light reflected back from the target tissue, (g) substantial tip vibration due to each pulse of light energy of a wavelength highly absorbed by water, such as from wavelengths of laser energy at 1400 to 1500 nm and 1800 to 2300 nm, which cause the almost instant conversion of water in the cells of the target tissue and any aqueous irrigation liquid to a steam bubble, whose expansion opposite the capillary tube disposed over the beveled, distal end surface of the optical fiber causes an equal and opposite force to be exerted against the capillary tube and the distal end of the side firing device, which can cause small or fatal cracks in the optical fiber and/or the capillary tube; (h) small cracks in the optical fiber and/or capillary tube created or expanded by the shock wave resulting from the collapse of the steam bubbles described in (g) above; (i) high levels of residual stress in the capillary tube caused by rapid thermal gradients when closing the distal end of the capillary tubing or fusing the capillary tubing to the glass cladding of the optical fiber, as it cools; and (j) softening and loss of flatness and/or integrity of the beveled distal end surface of the optical fiber due to thermal diffusion during fusion of the capillary tube to the glass cladding of the optical fiber.

Diminished transparency of the capillary tube can occur due to damage to and erosion of the capillary tube from various sources, including but not limited to laser energy reflected back from the target tissue, called “back reflected laser energy”, degradation of the capillary tube from tissue, blood and other bodily fluids back-splattered from the vaporization of tissue, which absorb laser energy and degrade the capillary tube, called “back splatter degradation”, reducing its laser energy transmission efficiency, and exposure of the capillary tube to hot gasses from the vaporization of tissue and, at wavelengths of laser energy which are highly absorbed by water, vaporization of the aqueous irrigation liquid commonly used in endoscopic procedures, causing erosion of the capillary tube, a process called “hydrothermal erosion”. If the capillary tube becomes sufficiently eroded or fractures, the air interface opposite the distal beveled end surface of the optical fiber may be lost, in which case the light will essentially be emitted straight ahead. In medical applications, this could cause unintended damage to a blood vessel, nerve or other tissue.

Lasers that emit wavelengths between about 190 nm to 1100 nm include for example, excited dimer or “excimer” lasers emitting at 193, 222, 248, 308 or 351 nm. Lasers emitting between about 400 to 1800 nm include, for example, argon lasers emitting at 488 to 514 nm, KTP lasers emitting at 532 nm, Nd:YAG lasers emitting at 1,064 nm, diode lasers emitting at various wavelengths from about 500 nm to 1800 nm, and a number of others. Lasers emitting at 1800-2300 nm include Thulium:YAG lasers emitting at 2000 nm and CTH:YAG lasers emitting at 2100 nm, generally called “Holmium” lasers, and others.

Diode and KTP lasers, emitting at least 80 watts of power, are able to vaporize tissue, albeit with some adverse effects, including charring (which prompts a healing response, causing pain and irritation for a few weeks), and inadvertent coagulation of deeper, unseen tissues, due to the light penetration and thermal diffusion to depths of 3 to 4 mm or more for these wavelengths in tissue, causing edema or swelling, which can take weeks to subside. When these lasers are used to vaporize a portion of the lobes of the prostate gland to treat BPH, residual coagulated tissue sloughs-off in the urine over a few weeks, causing pain and irritation for some weeks, and may cause periodic bleeding as the coagulated, dead tissue separates from live tissue.

As a result of the above adverse effects, pulsed lasers which emit in the 1800 to 2300 nm wavelength range, whose energy is highly absorbed by water, a constituent of virtually all living cells, have been effectively used for more efficient vaporization of tissue. Such lasers include typically emit laser energy in pulses with a duration of about 150 to 800 microseconds, typically at a pulse repetition rate of about 5 to 60 pulses per second, or alternately CW or quasi-CW, depending upon the application.

The light extinction or penetration depth of Holmium laser energy at a wavelength of 2100 nm is only about 0.4 mm in most tissues and the irrigation liquid cools the tissue between pulses, resulting in little or no charring and little thermal diffusion in tissue, resulting in less edema and swelling, and eliminating the inadvertent coagulation of or damage to unseen, deeper tissues. The steam bubbles created by vaporization of the irrigation liquid, when they cool, return to water vapor and ultimately to their liquid state.

For example, at a pulse repetition rate of 40 Hertz and a pulse width of 350 microseconds, there are 24,650 microseconds between each 350 microsecond pulse, for the irrigation liquid to cool the tissue between pulses, resulting in little or no charring of tissue.

Steam bubbles are almost instantly created upon each pulse of Holmium laser energy in an aqueous liquid field, like an explosion, as described briefly in subparagraph (g) above. As the steam bubbles expand, they cause an equal and opposite force to be exerted on the distal, laser energy emitting end of the bared optical fiber and capillary tube assembly, causing rapid movement, which can cause minute cracks to form and expand in the capillary tube, the distal end portion of the bared optical fiber and in the optical fiber near the optical fiber's fulcrum point.

When the steam bubbles collapse, they cause acoustic shocks, as briefly described in subparagraph (h) above, which can cause fractures in the capillary tube and the distal end portion of the bared optical fiber, diminishing their transmission efficiency. These acoustic shocks and the instant creation of steam bubbles occur with pulsed lasers from 5 to 50 or more times per second, resulting in rapid movement or vibration of the distal end of the side firing device. If such vibration causes the capillary tube or optical fiber to form cracks or break, light will escape in an aberrant direction, may damage unintended tissues and could harm the patient, operator or bystanders.

Commercially available side firing devices typically have an initial light transmission efficiency of only about 70 to 90%, which efficiency rapidly declines during use at higher laser powers (for example, at 50 Watts and above), and such side firing devices usually have the capability to vaporize only about 20 to 60 grams of soft tissue, such as the tissue of the male prostate gland, before failing or reaching an unacceptably low vaporization rate, which requires the side firing device to be discarded.

When designing a side firing device for medical applications, the outside diameter (“OD”) of a given device, consisting of the optical fiber, its attached capillary tube, and any optional outer tip or sheath should generally not exceed about 2.0 to 2.35 mm. If the distal end of the side firing device is optionally encased in a hollow, open-ended plastic or metal sheath, which protects the capillary tube from back splatter degradation and mechanical damage, with an opening or port for emission of the laser energy disposed 180° opposite the beveled, distal end surface of the optical fiber, the overall OD of the side firing device should generally not exceed about 2.0 to 2.3 mm. This is important, as endoscopes presently used in many surgical suites typically have an instrument channel with an inside diameter (“ID”) of 2.5 to 2.667 mm (7.5 to 8.0 French). While endoscopes with larger instrument channels are presently being marketed, physicians and hospitals prefer using their current inventory of endoscopes with smaller ODs, which are able to enter and pass through the urethra in the penis without damage to the urethra or to reach other tissues through small incisions or natural orifices.

A low core to glass cladding ratio of the optical fiber is generally less expensive and enables a thicker capillary tube to be used, whose greater thickness reduces the potential for damage from laser energy back reflected from the target tissue, back splatter degradation and hydrothermal erosion of the capillary tube, which can cause its premature failure, as described above. However, using a low core to cladding ratio subjects the thin, leading edge of the beveled, distal end surface of the optical fiber to damage, such as during assembly, transport and use, as described above, significantly reducing the transmission efficiency of the side firing device.

For example, a typical, commercially available 550 micron core diameter optical fiber, after the removal of its protective buffer coating and any polymer cladding from its distal end portion, has a core to cladding ratio of 1:1.1 and an O.D. of 600 microns, enabling a capillary tube with a wall thickness of about 500 microns to be disposed over the distal end portion of the optical fiber, bringing the O.D. of the combination to about 1650 microns or 1.65 mm. If an optional, hollow, protective plastic or metal sheath is used to encase the capillary tube, with a port for emission of laser energy as described above, with a wall thickness of about 300 microns, and allowing for a gap of 25 microns between the sheath and the capillary tube, the O.D. of the assembly will be about 2,300 microns or 2.30 mm.

Commercially available side firing fibers made by Lumenis, Ltd. (Yokneam Industrial Park, Israel) are used with Holmium lasers at a wavelength of 2100 nm, and commercially available side firing devices made by American Medical Systems Holdings, Inc. (Minnetonka, Minn.) are used with KTP lasers at a wavelength of 532 nm. Both of their side firing fibers have initial laser energy transmission efficiencies of about 75% to 90%, which decline relatively rapidly over time during their use, and are generally able to vaporize, at 100 or 120 watts of power, respectively, only about 20 to 60 grams of soft tissue, such as that of the male prostate gland, before declining in tissue vaporization efficiency to unacceptable levels or failing. These devices employ core to cladding ratios of about 1:1.1 and 1:1.4 or larger, respectively.

In many cases, reduced tissue vaporization rates during usage and failure of such side firing fibers from the loss of total internal reflection occurs. When this happens, laser light transmission must be immediately ceased by the surgeon to avoid damage to an unintended tissue, the failed device must be removed from the patient and discarded, and a new side firing fiber must be obtained from sterile storage, brought to the operating room, unpackaged, positioned in the patient and used to finish the treatment, substantially increasing the cost and time of the procedure.

It is an object of this invention to provide a side firing device which has a consistent, high laser energy transmission efficiency over 90%, with sufficient functional longevity (durability) and high reliability to enable it to efficiently and rapidly vaporize at least 90 grams of soft tissue prior to failure, such as the tissue of the male prostate gland, at a manufacturing cost as low as possible. Indeed, since single use, side firing devices sell for about $600 to $900 in the United States, if such a side firing device was sufficiently durable to vaporize at least 90 grams of soft tissue, it may be able to be cleaned, re-sterilized and used to remove sufficient tissue to treat, perhaps, three 30 to 50 gram prostates and, perhaps, two 60 to 90 gram prostates, significantly reducing the cost of the procedure.

### SUMMARY OF THE INVENTION

A side firing laser device suitable for use in medical and surgical procedures provides consistent, rapid vaporization of tissue and extended longevity. The device has a laser energy transmission efficiency in excess of 90%.

The side firing laser device includes an elongated conduit within which is situated an optical fiber with a beveled distal end capped by a closed end capillary tube carried by the conduit. The capillary tube is surrounded with an internally reflective sheath that defines a port through which laser energy emitted by the optical fiber exits.

In particular, the conduit has an open distal end to which is mounted a capillary tube that defines a cavity therewithin. The cavity is positioned to be in communication with the open end of the conduit. An optical fiber, adapted for coupling to a laser energy source, is provided in the conduit and terminates in a beveled distal end portion which freely extends into the cavity. The capillary tube is surrounded by a bulbous or rounded, internally reflective metal sheath which is also mounted to the conduit and which defines a port from which laser energy emitted by the beveled distal end portion of the optical fiber can exit. The sheath also defines an aperture which is aligned with the optical axis of the optical fiber.

### BRIEF DESCRIPTION OF THE DRAWINGS

FIG. 1 is an external, top view of a source of laser energy and a side firing optical fiber device constructed in accordance with the present invention.

FIG. 2 is a partial, cross-sectional, side view of the distal end portion of a side firing optical fiber device constructed in accordance with the present invention.

FIG. 3 is a partial, cross-sectional, side view of the proximal end portion of the shaft, handle, and other components utilized with a side firing optical fiber device constructed in accordance with the present invention.

FIG. 4 is a cross-sectional, end view at plane 4-4 in FIG. 2 of the side firing optical fiber device of FIG. 2.

FIG. 5 is a cross-sectional, end view at plane similar to 4-4 in FIG. 2 of a more preferred embodiment of the side firing optical fiber device of the present invention.

FIG. 6 is a partial side image of the distal end portion of a side firing optical fiber device constructed in accordance with the present invention, prior to use.

FIG. 7 is a partial side image of the distal end portion of a side firing optical fiber device constructed in accordance with the present invention, after 90 minutes of use.

### DETAILED DESCRIPTION OF THE PREFERRED EMBODIMENTS

While this invention is susceptible of embodiment in many different forms, there are shown in the drawings and will be described in detail herein specific embodiments thereof, with the understanding that the present disclosure is to be considered as an exemplification of the principles of the invention and is not to be limited to the specific embodiments illustrated.

To achieve the above mentioned objects of this invention and efficiently transmit at least 80 watts or more of laser power at, for example, a wavelength of 2100 nm from a Holmium laser, the components of the side firing device must be composed of and constructed to incrementally and consistently increase the laser energy transmission efficiency of the device, increase the rapidity and consistency of its tissue vaporization, extend its functional longevity, provide high reliability and provide optimal handling and positioning of the device.

These can be achieved by a series of features, entailing at least one of: (a) an optimal OD of the core of the optical fiber, allowing it to efficiently accept and transmit the amount and wavelength of laser energy desired, while being sufficiently small to allow the use of a cladding and thicker walled capillary tube; (b) an optimal wall thickness and composition of the glass cladding surrounding the core of the optical fiber, to minimize the size and cost of the cladding, which is typically the most expensive part of a glass optical fiber, (c) an optimal wall thickness of glass tubing collapsed over the glass cladding, to prevent excessive dispersion and damage to the thin leading edge of the beveled, distal end surface of the optical fiber during use; (d) an optimal OD of the bared optical fiber, which is sufficiently small to enable as large as possible wall thickness of a distally closed-ended capillary tube to be used to encase the beveled, distal end portion of the bared optical fiber; (e) an optimal bevel angle of the distal end surface of the optical fiber polished with a beveled angle slightly higher than would normally be predicted for total internal reflection (or “TIR”) given the numerical aperture and dispersion of the optical fiber materials using normal equations as known in the art, to reduce the transmission loss from the side firing optical fiber device by directing a higher percentage of the incoming laser energy laterally from its longitudinal axis; (f) an optimal flatness of the beveled, distal end surface of the optical fiber, to reduce losses which would reduce the side firing device's transmission efficiency; (g) an optimal thickness of the closed-ended capillary tube disposed over the beveled, distal end portion of the bared optical fiber, to maximize the device's functional longevity by increasing the capillary tube's resistance to damage and minimizing its susceptibility to hydrothermal erosion; (h) an optimal manner of attachment of the distally closed-ended capillary tube disposed over the beveled, distal end portion of the bared optical fiber, to maximize the side firing device's functional longevity by increasing the capillary tube's resistance to damage from back reflected laser energy, back splatter degradation and hydrothermal erosion during use; (i) an optimal means of fabricating the closed-ended capillary tube to optimize performance with respect to residual stresses remaining the capillary tube material(s) from thermally closing its distal end by thermal fusing and its cooling; (j) an optimal straightness and positioning of the bared distal end portion of the optical fiber to resist its being damaged during assembly with the capillary tube and during use; (k) an optimal protective sheath over the capillary tube with a port for emission of laser energy, to enable the capillary tube to resist the formation and/or propagation of minute cracks within the capillary tube from back reflection of laser energy, back splatter degradation and hydrothermal erosion, as described above; (l) an optimal protective sheath being fully or partially composed of a material highly reflective to the wavelength of laser energy being used, to reduce distal end losses by reflecting a higher percentage of both aberrant emissions within the device and reflected energy from the target tissue away from the longitudinal axis of the optical fiber, (m) an optimal hollow, relatively rigid shaft disposed over the optical fiber, proximal to the capillary tube, to enable the side firing device to resist the rapid movement or vibration resulting from the equal and opposite forces exerted on the distal end portion of the side firing device from the formation of steam bubbles and acoustic shocks occurring 5 to 50 times or more a second, as described above; (n) an optimal protective, lubricious plastic sleeve or coating disposed over the shaft encasing the optical fiber to allow the side firing device to be easily inserted into, used within and withdrawn from the working channel of commonly available endoscopes, without damage to the sleeve; (o) an optimal means to relieve pressure from the expansion of air or gasses trapped between the beveled, distal end surface of the optical fiber and the capillary tube, which are heated during lasing, to allow any gas pressure differential that may develop during use to be equalized; (p) an optimal means to fix the optical fiber in place, with respect to the capillary tube, to reduce stresses on the optical fiber during insertion of the side firing fiber into, during its use and its withdrawal from the side entry port of the instrument channel, often referred to as the “working channel” of an endoscope; (q) an optimal outside diameter of the side firing device to enable it to be used through the working channel of commonly available endoscopes; and (r) an optimal adhesive material to withstand the elevated peak and average temperatures created at the end of the side firing device during use, and which does not significantly absorb the wavelength of laser energy being used, causing it to melt and allow the adhesively bonded components of the side firing device to move with respect to each other or otherwise become degraded during use.

Common wisdom in the medical laser industry is that, to efficiently transmit 80 or more watts of power from a Holmium or similar pulsed laser or 80 or more watts of power from a diode or KTP laser, the highest possible initial laser energy transmission efficiency should be employed. Typically, this is done by beveling the distal end surface of the bared optical fiber and thermally fusing a distally closed-ended capillary tube to the bared, beveled, distal end portion of the optical fiber in the area of laser energy emission, by means known in the art. The reason for doing this is to eliminate the losses due to the glass-air-glass interface in the laser energy emission area caused by changes in the index of refraction, an approximately 3.5% transmission loss each time the index of refraction changes from glass to air to glass or an overall transmission loss of about 7%.

Closing the distal end of a capillary tube by thermal fusion typically causes stresses within the capillary tube to occur as it cools, weakening it, which can cause it to prematurely fail. Thermal fusion of the distal end portion of a capillary tube and fusion the capillary tube to a bared optical fiber is typically done with a carbon dioxide or CO₂ laser, an electric arc, a hydrogen flame or torch, or other means known in the art.

Also, thermal fusing of a distally closed-ended capillary tube to the bared, distal end portion of an optical fiber has many disadvantages. First, the fused portion of the bared, distal end portion of the optical fiber is mechanically fragile, which often requires a secondary means of securement of the capillary tube to the optical fiber, such as adhesively bonding the proximal end portion of the capillary tube to the proximal end portion of the bared, optical fiber.

Second, due to the capillary tube being fixedly attached to the optical fiber at more than one location, as the device rapidly vibrates during use (due to the equal and opposite mechanical forces from the formation and collapse of steam bubble during each laser pulse, as described above), the mechanically fragile optical fiber can fracture or become detached from the capillary tube, leading to additional losses and premature device failure.

Third, thermal fusing of the capillary tube to the bared, distal end portion of the optical fiber in the area of laser energy emission typically entails some loss of flatness of the beveled, distal end surface of the optical fiber due to its exposure to fusing temperatures above 1000° C. without exceeding the useful service temperature of the fiber's buffer (typically rated to between 200-400° C.) which in turn causes rapid cooling of the fused capillary tube and beveled distal end portion of the optical fiber. When these fused glass components rapidly cool, significant stresses are locked into the capillary tube and the optical fiber, which may cause them to become more susceptible to fracture and failure during use.

Fourth, any air or other gas trapped between the optical fiber and the capillary tube does not have the opportunity to escape when it becomes heated and expands during use, leading to pressure stresses being exerted on the beveled distal end portion of the optical fiber and the capillary tube, which can lead to their premature failure.

Although these fused devices can provide high in initial efficiency, some would fail after a few minutes of use at high laser power (for example, at 100 watts of Holmium:YAG laser power).

Contrary to conventional wisdom, we made several attempts to develop a side firing device which did not utilize thermally fusing the capillary tube to the bared distal end portion of the optical fiber, in order to overcome the aforementioned disadvantages of conventional fusing. We acted contrary to conventional wisdom in several respects denoted below.

First, we closed the distal end of the capillary tube by thermal fusion and annealed the capillary tube, allowing it to cool gradually at successively lower temperatures in a series of time-controlled steps, to reduce any residual stresses in the closed-ended capillary tube prior to its being disposed over the beveled, distal end portion of the optical fiber. Further, in some devices, we further tempered the outer surface of the capillary tube by rapid cooling, typically after the annealing cycle, to increase the strength in the outer surface of the closed-ended capillary tube prior to its being disposed over the beveled, distal end portion of the optical fiber.

Second, rather than fusing the capillary tube to the bared, distal end portion of the optical fiber in the area of laser energy emission, we close-fitted the capillary tube over the bared, distal end portion of the optical fiber, with a minimal gap between the optical fiber and the capillary tube not exceeding 40 microns, preferably about 1 to 25 microns, thus permitting the distal end portion to freely extend into the cavity of the capillary tube without any constraint. The length of distal end portion extending into the cavity of the capillary tube is at least two times the length of the bevel portion, which, of course, depends on the angle of the bevel. Stated in another way, the length of the distal end portion extending into the cavity is at least twice the ratio the optical fiber outside diameter to the value of the bevel angle tangent, i.e., 2×[optical fiber O.D./tan (bevel angle)].

Third, we close-fitted the capillary tube within the inner surface of a protective metal sheath, with a gap between the capillary tube and the inner surface of the protective metal sheath that is filled with adhesive. The gap usually does not exceed 40 microns, and preferably is about 1 to 20 microns.

The protective metal sheath disposed over the capillary tube, is preferably composed of very pure gold, silver, copper, or aluminum, preferably 99.5% pure silver (compared to sterling silver, which is 92.5% pure), which is highly reflective of the various wavelengths of laser energy commonly used through side firing fibers, or a thin film or strip of very pure gold, silver, copper, or aluminum is disposed over the capillary tube, beneath the protective metal sheath opposite its laser energy emitting area, which efficiently reflects aberrant laser beams from the distal, beveled distal end surface of the optical fiber and the inner and outer surfaces of the capillary tube disposed over the tip of the optical fiber, out of the laser energy emission port. For example, 99.5% pure silver reflects about 97.9% of 532 fun KTP laser output, 98.8% of 980 nm diode laser output and 98.9% of 2100 nm Holmium laser energy.

We also found by thermal imaging that heat generated by aberrant, forwardly emitted laser light could overheat the distal end of the protective metal sheath, causing damage to both the sheath and the capillary tube. To minimize likelihood of such damage the distal end of the protective metal sheath is provided with an aperture aligned with the optical axis of the optical fiber. If a Holmium laser is used, whose energy is highly absorbed by water, any laser energy escaping through the aperture in the sheath is harmlessly absorbed within a few millimeters (mm) of the irrigation fluid.

Fourth, to substantially reduce the rapid movement or vibration at the distal end of the side firing device and to better enable it to be held in position opposite the target tissue by the operator, we disposed a relatively rigid metal conduit or shaft over the optical fiber, including its buffer coating and any polymer cladding, from near the proximal end of the capillary tube to a point near the proximal end of a handle or handpiece for ease of use by the operator, substantially reducing rapid movement or vibration at the distal end of the side firing device from the equal and opposite forces exerted against the distal portion of the side firing device, as described above. The shaft or conduit can be metal or plastic, and preferably made of medical grade stainless steel. Preferably, the conduit has an ID of about 0.052″ and an O.D. of about 0.062″ for a wall thickness of about 0.005″ or about 125 microns.

The handle provides better control over positioning of the side firing device opposite the target tissue by the operator and can contain a visible and/or tactile button on its exterior, preferably positioned 180° opposite the direction of laser energy emission from the side firing fiber, as known in the art. When the operator's forefinger or thumb contacts the button, it points in the direction of laser energy emission. The handpiece or handle can be adhesively bonded to the exterior of the proximal end portion of the stainless steel shaft or secured by other means known in the art.

Fifth, we fixedly attached the optical fiber to the interior of the stainless steel shaft near its proximal end, proximal to the area of attachment of the handle or handpiece to the stainless steel shaft, enabling the bared, distal end portion of the optical fiber to move slightly within the capillary tube during insertion into and withdrawal from the side entry port of the working channel of the endoscope and during use. Securing the optical fiber to the stainless steel shaft, far away from the capillary tube, rather than securing the optical fiber to the capillary tube, leaving it free to move slightly when necessary within the capillary tube, prevented any localized stresses from occurring to the optical fiber both during transit through the side entry port of endoscopes and during use, which in prior experiments caused premature device failure during use.

Sixth, we created a channel or passageway within the stainless steel shaft to allow for expansion and contraction of air or other gasses trapped between the capillary tube and the distal end portion of the optical fiber, which are heated during laser emissions. In the event the channel within the shaft was not of adequate volume to equalize the pressure of such expanded air or other gasses, we provided a vent or opening in the stainless steel shaft near its proximal end, distal to the point at which the optical fiber is fixedly attached to the shaft, to allow any excessive volume of such gasses to escape into the atmosphere, outside the patient and the endoscope. We took special care that the fixed attachment method used to attach the optical fiber to the proximal end of the shaft terminated a sufficient distance proximally from the vent in the shaft to not obstruct the vent.

High speed photography of the side firing device during use indicated that there were occasions where gasses were seen to escape and on occasion contaminants were seen to burn up and escape from the space between the capillary tube and the optical fiber and exit out of the vent in the stainless steel shaft.

Seventh, to avoid the stainless steel shaft's scraping against the metal instrument channel of an endoscope, we encased the stainless steel shaft within a hollow, lubricious plastic sleeve, as described below, which was adhesively attached to the stainless steel shaft. We further crimped the proximal end of the reflective metal sheath over the distal end of the hollow, lubricious plastic sleeve, which was earlier disposed over the stainless steel shaft encasing the optical fiber, making the transition between the reflective metal sheath and the hollow, lubricious plastic sleeve as smooth as possible. If the transition between the proximal end of the reflective metal sheath and the hollow, lubricious plastic sleeve is rough, it can cause the proximal end of the reflective metal sheath to catch on a portion of the metal instrument channel of an endoscope upon removal of the side firing fiber from the endoscope, potentially causing the reflective metal sheath to partially or completely become dislodged from the distal end of the side firing fiber, causing the side firing device to fail.

As mentioned above, during testing of side firing devices, we found that entry and exit of the device from various commercially available endoscopes was a challenge when the stainless steel shaft rubbed against the stainless steel, side entry port to the working channels of these endoscopes, which ports may require the side firing device to pass through a 10° or greater entry angle. Further, some of these endoscopes had burrs where the side entry port joined the working channel, which caused devices without a smooth transition between the reflective metal sheath and hollow, lubricious plastic sleeve to have the reflective metal sheath become dislodged from the remainder of the device after multiple insertion/removal cycles.

To reduce the friction involved in these operations, a sleeve made of a lubricious and durable plastic, such as polyether ether ketone (PEEK) (Invibio, Inc., West Conshohocken, Pa.), with a wall thickness of about 125 microns can be provided to encase the stainless steel conduit or shaft. The side firing device can then be much more easily passed through and also resists scuffing when used in several commonly available endoscopes with a working channel of 2.5 mm (7.5 French) or larger, which allow sufficient space for infusing an irrigation liquid. Such endoscopes include those made by Karl Storz, Olympus, Richard Wolf, ACMI and others. The advantage of employing a thin tube of PEEK is its relatively low coefficient of friction and the ability to withstand scratching or scuffing when passing the side firing device into, moving it within during use and while removing it from the instrument channel of the endoscope a number of times.

When side firing devices were tested with an adhesive to (a) fixedly attach the proximal end of the capillary tube to the distal end of the stainless steel shaft, (b) fixedly attach the distal end of the capillary tube to the distal end of the reflective metal sheath, and (c) fixedly attach the proximal end of the reflective metal sheath to the distal end of the stainless steel shaft and lubricious and durable plastic tubing, the device had significantly more functional longevity than side firing devices tested with capillary tubes conventionally fused to the bared, distal end portion of the optical fiber in the area of laser energy emission, all other factors remaining constant.

When incorporating the above mentioned improvements, the adhesive utilized must survive the high peak temperatures during lasing (150° to 500° C. peak at the distal end of the side firing optical fiber device, determined by thermal imaging).

The adhesive must be able to resist such extremely high peak temperatures, be substantially transmissive of the wavelengths of laser energy used (e.g. from 300 nm to 2300 nm), and meet all sterilization and biocompatibility requirements of a medical device. Suitable adhesives are the optically transparent U.S.P. Class VI epoxide epoxy resins that exhibit at least 85 percent transmission in the range of about 400 to about 2500 nanometers and has a service temperature in air of at least about 100° C., preferably at least 120° C., more preferably about 135° C. Preferably, the adhesive has an average air service temperature of about 135° C., is substantially transparent to laser energy at 300 to 2500 nm and has a pull strength, after curing, of above 20 lb-f. with a 125 micron bond gap. A particularly preferred adhesive absorbs only about 6% of KTP laser energy, 6% of diode laser energy at 980 nm and 6% of CTH:YAG or Holmium laser energy.

When tested in a water bath with the distal end of a 550 micron core fiber placed in contact with the side wall of a capillary tube with both ends sealed with a strip of very pure silver with a wall thickness of 250 microns coated with this adhesive adhered to the back side of the capillary tube, when 10 Watts of Holmium Laser power was emitted at 1 joule per pulse at 10 pulses per second for 10 minutes, there was no visible degradation of the adhesive or the underlying silver strip.

When side firing devices employing the aforementioned improvements were tested, their transmission efficiency and tissue vaporization rate were consistently higher and their functional longevity was significantly longer lasting, compared to current commercially available side firing devices.

Sixty side firing devices embodying the present invention were tested, and they were able to consistently achieve without failure at least 90 minutes of continuous laser emission at 80 to 135 watts of Holmium:YAG laser input power.

Further, and as significant, these side firing devices did not significantly decline in laser transmission efficiency and tissue vaporization rate, compared to commercially available side firing devices.

We conducted a series of experiments with various optical fibers to minimize losses. We discovered that a Low-OH optical fiber with a core diameter of about 450 microns could accept and transmit up to 150 watts of Holmium laser power. Optical fibers with larger core-to-clad ratios were found to have less overall losses than those with smaller core to clad ratios (e.g. 1:1.2 vs. 1:1.1).

Increasing the fiber core to cladding ratio is relatively expensive, as fiber cladding in glass fibers is typically made by fluorinating the fused silica of the cladding to create a lower index of refraction than that of the core of the optical fiber. However, substantially the same difference in the index of refraction between the outer glass layer of the fiber and the environment outside the optical fiber can be achieved by other means. We experimented with optical fibers with a very small core to clad ratio (e.g. 1:1.05) with an undoped thicker outer layer or “overjacket” of fused silica over the thinner, fluorinated, fused silica cladding, made as part of the overall optical fiber drawing process. This unique optical fiber construction is less expensive than conventional optical fibers used in medical applications, due to the thinner layer of fluorinated glass cladding, and provides the added benefit of a slightly larger index of refraction difference between the outer, undoped, fused silica overjacket of the optical fiber and the environment, which, allows a slightly higher angle of reflection from the beveled, distal end surface of the optical fiber. When testing side firing fibers of this construction, we found that the lateral or side-firing initial transmission efficiency was slightly better than conventional optical fibers with full thickness fluorinated cladding, at a fraction of the cost.

During our experiments, we found that there was an optimal core to overall combined fluorinated cladding and undoped, fused silica overjacket ratio to obtain the advantages of this approach, without sacrificing either the amount of space remaining available for a greater wall thickness of the capillary tube, or the space available for a protective, metal sheath over the capillary tube, to avoid the outside diameter of the side firing device exceeding 2.3 mm and not fitting within the 2.5 mm or larger instrument channel of many, commonly available endoscopes, leaving a sufficient space for infusing an irrigation fluid.

To achieve optimal laser energy transmission efficiency, we discovered that the optimal core to overall combined fluorinated fused silica cladding and undoped fused silica overjacket ratio was about 1:1.25 to 1:1.39, preferably about 1:1.34. Further, we found that if the core to outer glass size ratio is greater than about 1:1.4 with a fiber core size large enough to transmit up to 150 watts of Holmium:YAG laser power, the capillary tube cannot have a sufficiently large wall thickness to endure the above described hydrothermal erosion for a sufficient period of time (e.g. 60 minutes or longer), without increasing the O.D. of the device beyond about 2.17 mm.

According to prior art, for efficient transmission of laser energy, the core to cladding ratio (not core to overall glass size) of the optical fiber should be 1:1.4 or higher. However, due to the relative mechanical weakness of the fluorinated glass cladding, as compared to undoped synthetic fused silica, a side firing angle at or below that which would normally be needed for total internal reflection so that greater than about 90% of electromagnetic radiation reflected by the reflecting surface is incident on the particular area at below a critical angle for transmission through the transmitting surface in the lateral direction, inadequate wall thickness of the capillary tube disposed over the beveled, distal end portion of the optical fiber, and its reduced resistance to back reflected laser energy, back splatter degradation and hydrothermal erosion, the commercially available side firing devices achieve an overall laser energy transmission efficiency of only about 90% and sometimes fail before vaporizing 30 to 80 grams of soft tissue, such as that of the male prostate.

We discovered that the thin fluorinated fused silica cladding, surrounded by an undoped fused silica overjacket, as described above, enabled the optical fiber to be beveled at an angle greater than the critical angle for total internal reflection from standard equations known in the art, so that over 90% of the laser energy is reflected by the beveled, distal end surface of the optical fiber.

When testing different angles of distally beveled optical fiber surfaces, we found that optical fibers of different construction had different efficiency curves. While some fibers with smaller core to clad ratios performed very close to the theoretical maximum angle for TIR (for the 2.1 μm wavelength with 0.22 NA Low-OH fibers, the maximum theoretical angle for TIR is 37°), optical fibers with larger core to clad ratios, using the previously described thin, fluorinated, fused silica cladding surrounded by a thicker, undoped, fused silica overjacket, performed differently from theory. For these types of side firing fibers, the maximum theoretical angle for TIR could be exceeded with an increase in overall laser energy transmission efficiency, due to their outer layer or overjacket of undoped fused silica having a slightly higher index of refraction than a typical optical fiber with a customarily thick and more expensive fluorinated glass cladding.

Using such undoped fused silica overjacketed optical fibers on top of a thin cladding of fluorinated fused silica, we tested side firing fibers whose distal ends were beveled and mechanically polished at an angle of between 35° and 42° relative to the optical axis of the optical fiber in 1° increments with a Holmium laser. We found that their laser energy transmission efficiency improved from about 84% at 35° and increased each degree up to about 95% at 41°, but rapidly decreased in efficiency to below about 84% at an angle of 42° or greater.

To maximize laser energy transmission efficiency, the beveled, distal end surface angle is optimized for a combination of a given laser's wavelength, numerical aperture, cladding, and other optical fiber constructional details, and is closely controlled. Thus, for the following experiments with the same Holmium laser and side firing fiber construction, we maintained the beveled, distal end surface of the optical fiber at an angle of between 40° and 41°, resulting in the emission of laser energy, according to Snell's Law, at an angle of about 80° to 82° from the axis of the optical fiber.

We then experimented with determining the magnitude of the different losses at different spacial orientations from the distal end of devices with the aforementioned 40° to 41° beveled, distal end fiber surface and our previously described combined cladding and overjacket construction. We found that while the majority of losses were directed opposite the primary, side firing emission direction, there were additional smaller losses directed to the left and right sides of the primary, side firing emission direction, as well as smaller losses directed both forward and backward along the longitudinal axis of the optical fiber. In investigating these losses, we found that the flatness of the beveled, polished, distal end surface of the optical fiber can have a significant impact on the magnitude of these losses.

When testing the flatness of the beveled, distal end surfaces of optical fibers with a Michelson interferometer, utilizing a 635 nm diode laser, we discovered that the aberrant reflections or losses, although relatively small, could be reduced by over 60% by decreasing the measured curvature of the beveled, distal end surface of the optical fiber from 5.3 microns to 1.3 microns. This increased flatness allows more consistency in laser transmission efficiency among all devices of the same configuration, and is easily determined by measuring surface flatness directly or by measuring the losses from the sides of the fiber and ensuring that they were below a certain percentage, which can be equated to a specific surface flatness.

During the construction of various side firing devices, we noticed that the distal, leading edge of the optical fiber's beveled surface was fragile and susceptible to damage during assembly, subsequent handling and use. We call the thin leading edge of the beveled, distal end surface of the optical fiber the “tip”, and we call the thickest portion of the beveled, distal end surface of the optical fiber the “root”. We also discovered that, as optical fibers in the drawing process are traditionally wound and stored on a roll or spool, when the optical fiber is un-spooled, it has taken a slight set, so it is not quite straight when laid naturally on a flat surface. We were able to take advantage of this fiber set to reduce the susceptibility of the tip to damage by orienting the fiber during polishing and assembly, so that the tip is aligned with the section of the fiber furthest away from the center of the bend radius (e.g. the very outside portion of the fiber set) and the root is aligned with the section of the fiber closest to the center of the bend radius (e.g. the very inside portion of the fiber set).

By using this technique, we discovered that, when the tip is inserted into the capillary tube, the root tends to rest against the inner surface of the capillary tube opposite the area of laser transmission, with a small gap between the tip and the inner surface of the capillary tube through which light is transmitted. This prevents significant stresses on the tip that are present and detectable with either a microscope or a polarimeter, compared to when the opposite of the above positioning technique is used. This has also substantially reduced the occurrence of chips at the tip of the fiber during manufacture, handling, and use.

As one of the objectives of this invention is a highly durable side firing device, with the longest possible functional longevity, the material and dimensions of the capillary tube are significant. During our testing, we found that natural fused silica capillary tubes did not work as well, and their consistency and durability was not as desirable, as natural fused silica has a certain level of natural defects. If those defects are present in the portion of the capillary tube through which laser energy is transmitted, the device had shorter functional longevity and lower reliability.

We found that the use of synthetic fused silica capillary tubes, formed by inside or outside vapor deposition, enabled the side firing device to achieve enhanced longevity. Further, we discovered that fluorinated, synthetic fused silica capillary tubes, instead of fluorinated natural fused silica, can be used to further increase the longevity of the side firing device, as fluorinated, synthetic fused silica reduces the rate at which hydrothermal erosion occurs in the capillary tube during use, with little increase in device cost, due to their short length.

From a dimensional standpoint, we found as the wall thickness of the capillary tube in the area through which laser energy is transmitted was increased, the functional longevity of the capillary tube and the side firing device was increased. Upon testing capillary tubes of both fluorinated and unfluorinated synthetic fused silica with various wall thickness, we also discovered that, to enable the capillary tube encasing the beveled, distal end portion of the optical fiber to best endure hydrothermal erosion, back splatter degradation and back reflection of laser energy, the capillary tube should have a wall thickness of at least about 500 microns in the area through which laser energy is transmitted.

Thus, within the overall size constraints of the device, minimizing the ID of the capillary tube (e.g. 650 microns, including the approximately 25 micron gap described above) and maximizing its OD (e.g. 1650 nm or 1.65 mm), enabled us to utilize a capillary tube with a 510 micron wall thickness to resist the above-described damage, after encasing the capillary tube with an optional, hollow, open-ended metal sheath with a wall thickness of 300 microns with a port for emission of the laser energy, as described above, for an overall device OD of 2.3 mm, for a device with a high functional longevity and high reliability, all other factors remaining constant.

Capillary tubes can be formed with an eccentric inner hollow bore or channel, without changing their outer, circular diameter. These capillary tubes allow the wall thickness in the portion through which laser energy is transmitted to be significantly larger (e.g. up to about 750 microns) than the wall thickness in the portion opposite laser emission (e.g. as little as about 270 microns), which allows increased functional longevity of the side firing device, due to the thicker wall of the capillary tube in the path of laser energy emission being more resistant to hydrothermal erosion, back splatter degradation and back reflection of laser energy, where all other factors remain constant.

In laboratory bench testing, thirty (30) side firing devices were constructed in accordance with the present invention, as described herein with the optical fiber having a core diameter of about 450 microns, encased by a thin, fluorinated, fused silica cladding and an undoped, synthetic fused silica overjacket, with a combined cladding and over-jacket wall thickness of about 75 microns, for a core to overall glass size of about 1:1.34, disposed within a distally closed-ended capillary tube of synthetic fused silica, with a wall thickness of 510 microns, with a gap of about 28 microns between the exterior of the optical fiber and the interior of the capillary tube, covered by a highly pure silver, hollow, open-ended metal conduit or sheath with a wall thickness of 300 microns, with a gap of about 20 microns between the exterior of the capillary tube and the interior of the silver sheath, for an overall O.D. of the side firing optical fiber device of about 2.3 mm.

When 100 or more watts of Holmium:YAG laser power was transmitted through the above described devices, using a automated robotic arm to move the side firing device at a uniform speed at a uniform position above soft animal (porcine) tissue in a water bath, their initial transmission efficiency averaged 91%, they were able to vaporize an average of 3.19 grams of soft animal tissue per minute over a period of 90 minutes, and they were all functioning satisfactorily after 90 minutes of lasing, with little reduction in tissue removal rate, no cracking or failure of the capillary tubing, no tip dislodgement, or other malfunctions, and with only normal expected device degradation.

Thirty (30) devices of the same construction were tested on the same Holmium laser under the same conditions by an independent testing laboratory, whose testing confirmed the above-described results.

In actual use by a surgeon manually in the prostate of a patient, the tissue vaporization rate is expected to be substantially lower, about 1.5 grams per minute, over a period of about 90 minutes, as Holmium laser power is lost vaporizing irrigation liquid if the side firing device is held too far away from the target tissue and, if the side firing device is held so that it touches the target tissue, the capillary tube can be contaminated and degraded.

Further, these features enable a side firing device constructed as described above to be cleaned after use, steam sterilized and reused, perhaps several times, significantly reducing its cost to hospitals, surgery centers and others.

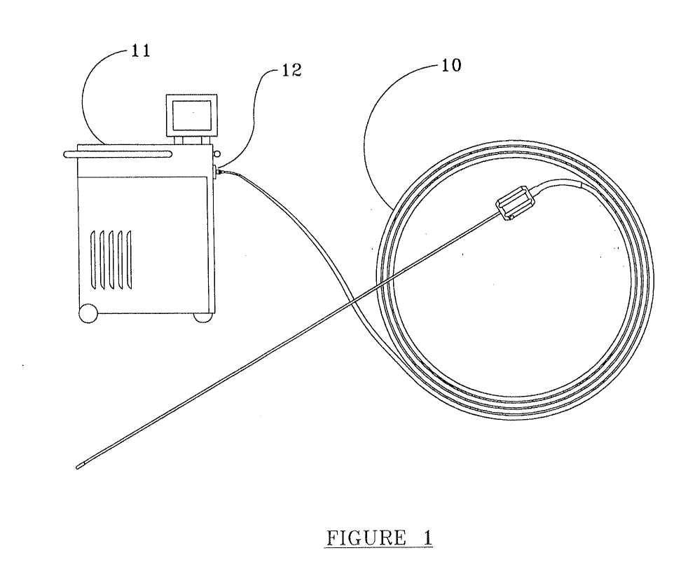

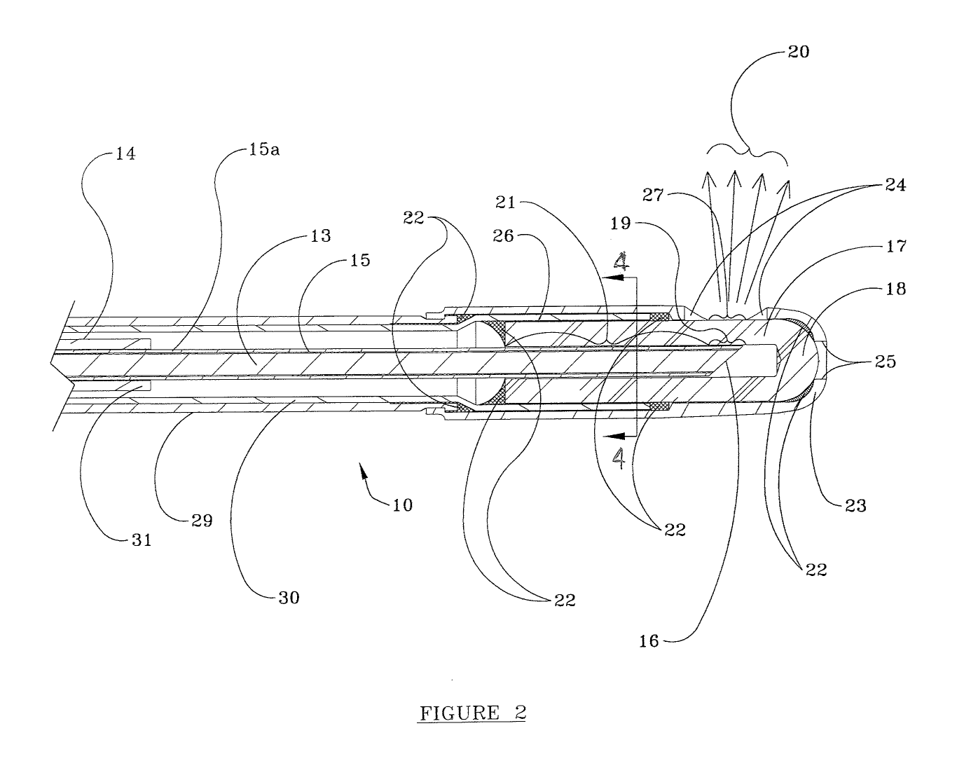

As shown in FIG. 1, side firing device 10, constructed in accordance with the present invention, utilizes a source of laser energy 11, which transmits its laser energy into optical coupler 12, which focuses and delivers the laser energy into the proximal end of fused silica core 13 (shown in FIG. 2) of optical fiber 14, which extends to near the distal end of side firing device 10. Laser energy source 11 generates whatever wavelength of laser energy is desired, including without limitation an excimer, KTP, diode, Nd:YAG, Thulium:YAG, or Holmium:YAG laser, preferably a Holmium:YAG laser at a wavelength of about 2100 nm.

As shown in FIG. 2, optical fiber 14 of side firing device 10 of the present invention has a fused silica core 13 with a diameter of about 450 microns and a refractive index of about 1.461 at 532 nm, 1.451 at 980 nm, about 1.450 at 1064 nm, about 1.439 at 1900 nm and, preferably, about 1.437 at about 2100 nm. Core 13 is surrounded by a thin, fluorinated, fused silica cladding 15 with a thickness of about 11 microns with a sufficiently lower refractive index to cause total internal reflection of light, with a core 13 to cladding 15 ratio of about 1:1.05.

Fluorinated, synthetic fused silica cladding 15 is encapsulated by an outer overjacket 15a of undoped, synthetic fused silica, with a wall thickness of about 65 microns, which brings the actual glass outer diameter (O.D.) of optical fiber 14 to about 600 microns, and the combined cladding 15 and overjacket 15a bring the core 13 to overall combined fluorinated fused silica cladding 15 and undoped fused silica overjacket 15a ratio to about 1:1.34. The distal end portion of outer overjacket 15a of optical fiber 14 was earlier stripped of any polymer cladding and protective buffer coating 31.

The distal end of optical fiber 14 has distal end surface 16 with a bevel angle of 40° to 41° relative to the optical axis of the fiber. The beveled, distal end portion of optical fiber 14 is disposed within a cavity of distally closed-ended capillary tube 17, which has a wall thickness of 100 to 1000 microns, preferably about 510 microns. Capillary tube 17 can be made of natural fused silica, but is preferably made of synthetic fused silica to reduce imperfections and increase laser efficiency and durability, and is most preferably made of fluorinated, synthetic fused silica to increase its resistance to back splatter degradation and hydrothermal erosion when used in a liquid environment. Undoped fused silica overjacket 15a and capillary tube 17 each have wall thicknesses sufficient to endure a significant amount of use, prevent chipping of undoped fused silica overjacket 15a, mechanical damage, laser energy reflected back from the target tissue, back splatter degradation and hydrothermal erosion. Capillary tube 17 is not attached to, i.e., is spaced from, undoped, synthetic fused silica overjacket 15a of optical fiber 14.

Capillary tube 17 has a closed distal end 18. This is accomplished by thermally fusing the distal end of capillary tube 17 to close its distal end 18, using CO₂ laser energy, an electric arc, a hydrogen flame or other means known in the art that ensure no contaminants are introduced into the capillary tube 17 during this process. After closing the distal end 18 of capillary tube 17, capillary tube 17 is further processed by annealing using a series of small, timed reductions in temperature from its maximum dwell temperature of about 1200° C. by about 50° C. each over a period of about 20 minutes each, to avoid the formation of residual stresses in capillary tube 17.

Capillary tube 17 is close-fitted over the beveled, distal end portion of bared optical fiber 14 with a gap between the fiber and sidewall of capillary tube 21 not exceeding 40 microns, preferably about 1 to 25 microns. Gap between fiber and capillary tube 21 is not filled with an adhesive or other material, but is left open. Gap between fiber and capillary tube 21 allows air, other gasses or materials trapped between closed distal end 18 of capillary tube 17 and beveled, distal end surface 16 of optical fiber 14, which are heated during the emission of laser energy, to expand and not over-pressurize and damage capillary tube 17 or optical fiber 14.

Laser energy is laterally emitted from beveled, distal end surface 16 of optical fiber 14 by total internal reflection, due to capillary tube 17 providing an air interface with a lower index of refraction than that of core 13 of optical fiber 14 opposite beveled distal end surface 16 of optical fiber 14. The laser energy passes through laser energy emission area 19 of capillary tube 17, and exits as shown by arrows 20 at an angle of about 80° to 82°.

Capillary tube 17 is close-fitted within internally reflective metal sheath 23, which is preferably made of very pure gold, silver, or another reflective metal, most preferably 99.5% pure silver (for comparison, sterling silver is 92.5% pure). Metal sheath 23 is highly reflective of the wavelength of laser energy typically used through side firing device 10. For example, 99.5% pure silver reflects about 98.9% of Holmium laser energy at 2100 nm, about 97.9% of KTP laser energy at 532 nm and about 98.8% of diode laser energy at 980 nm. Optionally, reflective metal sheath 23 may be made of a metal coated with a dielectric or other coating known in the art which is highly reflective to the wavelength of light being transmitted.

Reflective metal sheath 23 has a wall thickness of 10 to 1000 microns, preferably about 300 microns and is close-fitted over capillary tube 17. Gap between capillary tube and reflective metal sheath 26 is no more that 40 microns, preferably about 10 to 20 microns, and is filled with an adhesive 22. Protective metal sheath 23 protects capillary tube 17 from mechanical damage, laser energy reflected back from the target tissue, back splatter degradation and hydrothermal erosion, which results in an overall O.D. of side firing device 10 of about 2.3 mm, and enables it to pass in and out of 2.5 mm (7.5 French) or larger diameter instrument channels of commonly available endoscopes. The inner surface of reflective metal sheath 23 also reflects aberrant beams of laser energy from imperfections in beveled surface 16 of optical fiber 14 and capillary tube 17 back through optical fiber 14, most of which pass out of laser energy emission port 24 in protective metal sheath 23.

Capillary tube 17 is not fused or otherwise fixedly attached to fused silica overjacket 15a of optical fiber 14 at area 19 or at any other location. No adhesive 22 is used to attach capillary tube 17 to fused silica overjacket 15a of optical fiber 14 at gap 21 or any other place. Instead, capillary tube 17 is positioned during assembly and close-fitted within hollow conduit 30 and reflective metal sheath 23, and is fixedly attached to conduit 30 and sheath 23 by adhesive 22.

Laser energy emission port 24 in reflective metal sheath 23 is disposed over the region of laser energy emission 19 of capillary tube 17, which is disposed 180° opposite the distal, beveled end surface 16 of optical fiber 14. The distal end of reflective metal sheath 23 is provided with an aperture 25 to permit any forwardly emitted laser energy to exit without overheating metal sheath 23 or capillary tube 17. Aperture 25 can also serve as a detection system of capillary tube failure by the user if the forwardly emitted laser energy substantially and quickly increases during use. As shown, the distal end of metal sheath 23 preferably is bulbous or rounded, but it can be blunt, conical, sharp, syringe-needle shaped, trocar shaped or of any other desired shape.

To prevent mechanical damage to optical fiber 14 proximal to the proximal end of capillary tube 17, hollow conduit 30 is disposed over optical fiber 14 to resist the equal and opposite forces exerted upon the distal end portion of side firing device 10 by the emission of laser energy, preferably by forces exerted by steam bubbles formed by the emission of Holmium laser energy at a wavelength of 2100 nm in an aqueous liquid field and the resulting acoustic shock from the collapse of said steam bubbles as described earlier, enabling the operator to better maintain the side firing device in position opposite the target tissue during use.

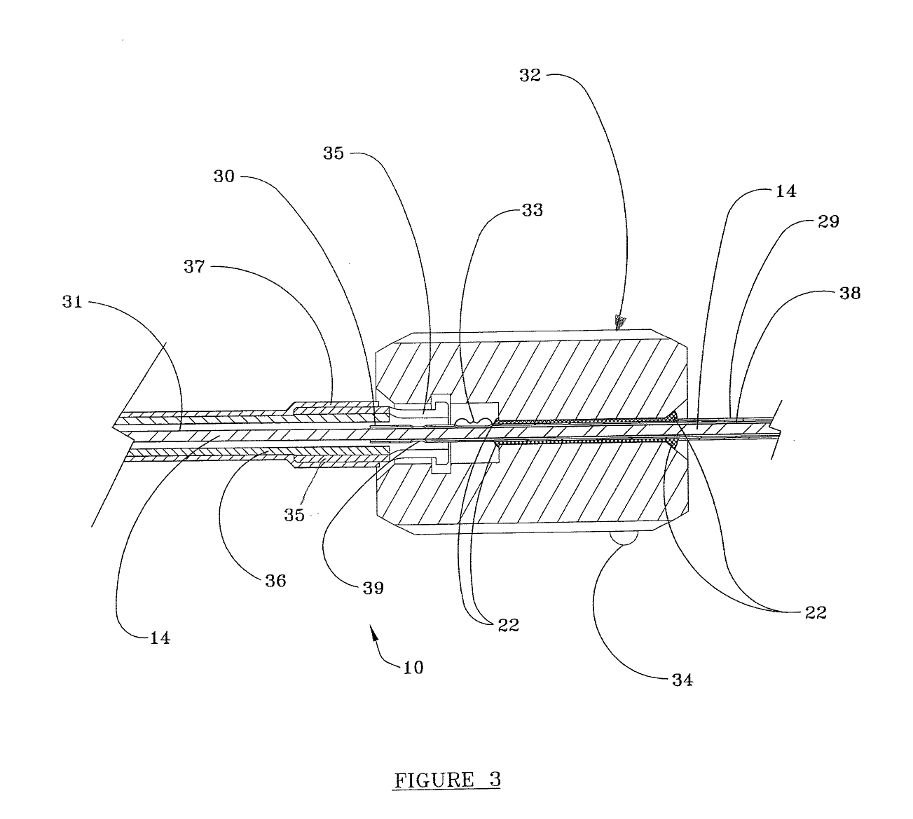

As shown in FIG. 3, hollow plastic or metal shaft 30 is fixedly attached by adhesive 22 to the interior of handle 32 near its proximal end. Shaft 30 is also fixedly attached to optical fiber 14 near its proximal end by one or more crimps 39, by both adhesive 22 and one or more crimps 39, or by other means known in the art.

To improve the ease of passing side firing device 10 through the instrument channel of commonly available endoscopes (not shown), some of which may have a side-entry port (not separately shown), which may intersect with the instrument channel of the endoscope at an angle of about 10° or more, and may have burrs or other imperfections where the side-entry port intersects with the instrument channel, hollow plastic or metal shaft 30 can be coated with a thin film or covered by a hollow, plastic outer sleeve 29, which may be made of a lubricious material, such as Fluorinated Ethylene Propylene (Teflon® FEP) or Ethylene Tetrafluoroethylene (Tefzel® ETFE), made by the DuPont Co. of Wilmington, Del., or, preferably, to resist scuffing, lubricious plastic sleeve 29 is preferably made of a durable and lubricious plastic, such as PEEK (Invibio, Inc., West Conshohocken, Pa.). Plastic lubricious sleeve 29 is fixedly attached to hollow plastic or metal shaft 30 by adhesive 22 or other means known in the art.

About half of the proximal end of capillary tube 17 is fixedly attached to the distal end of shaft 30 by adhesive 22. To maintain the integrity of and prevent the unwanted dislodgement of reflective metal sheath 23 during removal from endoscopes, the proximal end of reflective metal sheath 23 is swaged or otherwise collapsed over the distal end of lubricious plastic sleeve 29, to allow for a smooth mechanical transition between lubricious plastic sleeve 29 and reflective metal sheath 23.

To resist the equal and opposite forces on the distal end portion of side firing device 10 from steam bubble explosions and photo acoustic shock waves, as described earlier, hollow metal or plastic shaft 30 extends over optical fiber 14, from near the middle of capillary tube 17 (as shown in FIG. 2) to near the proximal end of handle 32.

Handle 32 may be fixedly attached to the exterior of hollow plastic or metal shaft 30 by adhesive 22. Handle 32 may optionally be attached to the exterior of metal shaft 30 and moveable (not separately shown) with respect to the portion of optical fiber 14 extending from the proximal end of protective metal sheath 23 to the source of laser energy 11, by means known in the art. Handle 32 optionally has a fixed orientation knob 34 positioned on handle 32, preferably positioned 180° opposite the beveled, distal end surface 16, and opposite the area of laser emission 27, to allow the user's finger or thumb, when placed on the orientation knob 34, to point in the direction of laser emission.

Hollow metal shaft 30 is fixedly attached to buffer coating 31 of optical fiber 14, proximal to vent opening 33 in shaft 30, by one or more crimps 39 in shaft 30 to buffer coating 31 of optical fiber 14, by adhesive 22, or by other means known in the art. To allow any air or gasses trapped between beveled, distal end surface 16 of optical fiber 14 and capillary tube 17, which are heated and expanded during the transmission of light, to escape into the atmosphere and avoid a build-up of pressure between these components, hollow metal or plastic shaft 30 has channel 38 extending proximally from the proximal end of capillary tube 17 to opening or vent 33, distal to the attachment of optical fiber 14 to the interior of metal or plastic shaft 30 by one or more crimps 39, adhesive 22, and the like.

In addition to shaft 30 resisting rapid movement or vibration of side firing device 10, the presence of hollow shaft 30 also makes it easier for the operator to maintain the distal end of side firing device 10 at a desired position opposite the target tissue during use.

To protect optical fiber 14 as it exits the proximal end of handle 32 from excessive bending or breakage, and to allow ease of rotation of area of laser energy emission 27 within port 24 in protective metal sheath 23, snap collar 35 is inserted into the proximal end of handle 32 during assembly. Prior to insertion of snap collar 35 into the proximal end of handle 32, snap collar 35 is attached to the distal end of protective jacket 36 and strain relief 37 by adhesive 22, or the like.

Protective jacket 36 has a wall thickness of about 700 microns and extends about 100 microns from within the proximal end of handle 32 over strain relief 37. Strain relief 37 has a wall thickness of about 400 microns and extends from within the proximal end of handle 32 and is about 10 cm in length. Protective jacket 36 and strain relief 37 protect optical fiber 14 from being bent excessively as it enters handle 32 and hollow shaft 30, as known in the art. Protective jacket 36 also protects optical fiber 14, including buffer coating 31 on optical fiber 14, during use by preventing clamping of optical fiber 14 to surgical drapes, gowns, and the like, and prevents damage to optical fiber 14 during handling before or after use of side firing device 10.

Reducing the diameter of core 13 of optical fiber 14 to 450 microns, from the customary 550 to 600 micron core diameter of optical fibers commonly used in side firing devices made by others, results in an increase in the energy density or fluence of the emitted laser energy beam, as shown by arrows 20. For example, at 2 joules per pulse at 50 Hz (100 watts) of Holmium laser energy, the fluence from the area of laser emission 27 of optical fiber 14 is 23.6 kW/cm², compared to 18.0 kW/cm² if the diameter of core 13 of optical fiber 14 was 550 microns and the same 100 watts of Holmium:YAG laser energy was transmitted through optical fiber 14.

Similarly, if 180 watts of KTP (532 nm) laser power is used, the power density of the emitted laser beam with a core 13 of optical fiber 14 having a diameter of 450 microns from the area of laser emission 27 is 42.5 kW/cm², compared to 32.4 kW/cm² if the diameter of core 13 of optical fiber 14 was 550 microns and the same 180 watts of KTP (532 nm) laser power was transmitted through optical fiber 14. Furthermore, if 300 watts of diode (980 nm) laser power is used, the power density of the emitted laser beam with a core 13 of optical fiber 14 having a diameter of 450 microns is 70.8 kW/cm², compared to 54.0 kW/cm² if the diameter of core 13 of optical fiber 14 was 550 microns and the same 300 watts of diode (980 nm) laser power was transmitted through optical fiber 14.

The laser light transmission efficiency of side firing device 10, constructed in accordance with the preferred embodiment of the present invention, is at least about 91% when used with Holmium laser energy, about 90% when used with KTP (532 nm) lasers and about 91% when used with diode (980 nm) lasers.

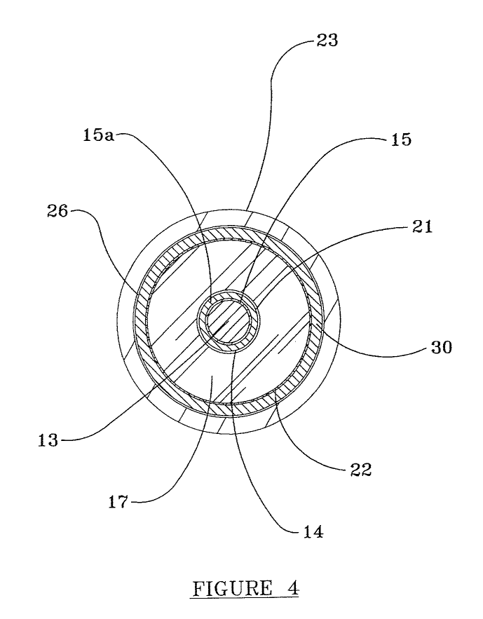

As can be seen in FIG. 4, optical fiber 14 is comprised of core 13, which is preferably made of fused silica with an O.D. of about 450 microns, surrounded by a preferably fluorine-doped fused silica cladding 15, more preferably synthetic fused silica, which has fewer imperfections and impurities than natural fused silica. Cladding 15 has a wall thickness of about 11 microns and an O.D. of about 472 microns, and outer overjacket 15a, of preferably undoped fused silica, most preferably undoped, synthetic fused silica, has a wall thickness of about 65 microns and an O.D. of about 600 microns.

Capillary tube 17, which is made of undoped fused silica, preferably undoped, synthetic fused silica and most preferably of fluorine-doped, synthetic fused silica, as synthetic fused silica has fewer imperfections and impurities than natural fused silica. Furthermore, fluorinated, synthetic fused silica has higher resistance to back reflected laser energy, back splatter degradation and hydrothermal erosion. Capillary tube 17 is close-fitted over the bared, distal end portion of optical fiber 14 (after any optional polymer cladding and buffer coating is earlier removed), with gap 21 between capillary tube 17 and optical fiber 14 not exceeding about 40 microns, preferably about 1 to 25 microns.

Capillary tube 17 has a wall thickness of about 510 microns and an O.D. of about 1650 microns. Capillary tube 17 is also close-fitted within the inner surface of hollow, reflective metal sheath 23, which has a wall thickness of about 300 microns, with gap 26 between capillary tube 17 and metal sheath 23 not exceeding about 40 microns, preferably about 10 to 20 microns. Gap 21 is not filled with an adhesive, while gap 26 is filled with adhesive 22.

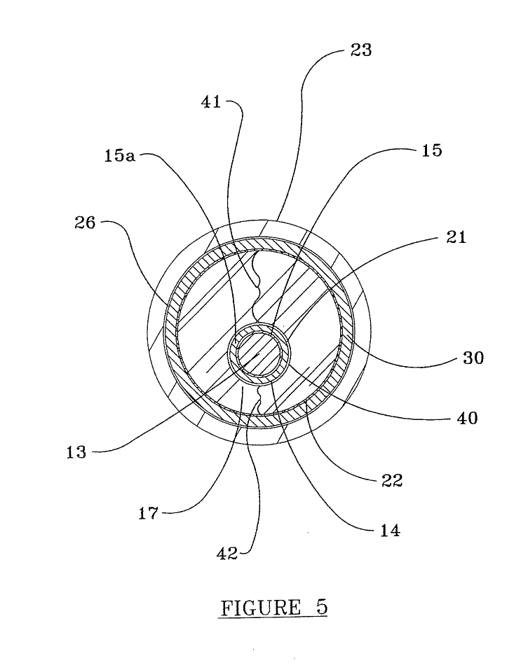

As shown in FIG. 5, capillary tube 17 has the same O.D. as capillary tube 17 shown in FIG. 4. In this more preferred embodiment of device 10 of the present invention, capillary tube 17 has eccentric channel 40, which is oriented to provide the greatest wall thickness 41 of capillary tube 17 at the area of laser energy transmission 19 from capillary tube 17, to provide added protection to capillary tube 17 from back reflection of laser energy, back splatter degradation and hydrothermal erosion, as described earlier. The relatively thinner wall thickness 42 of capillary tube 17 can be seen at the bottom of reflective metal sheath 23.

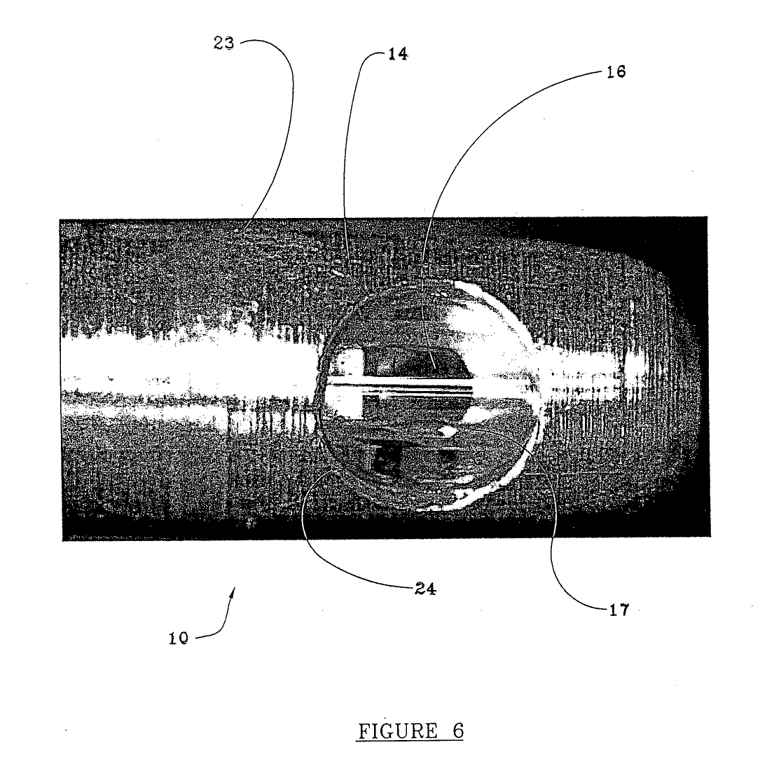

As seen in FIG. 6, the distal end of side firing device 10 shows reflective metal sheath 23, port 24 in sheath 23, capillary tube 17, optical fiber 14, and beveled distal end surface 16 of optical fiber 14. This image was taken prior to any use of laser energy through device 10 on tissue.

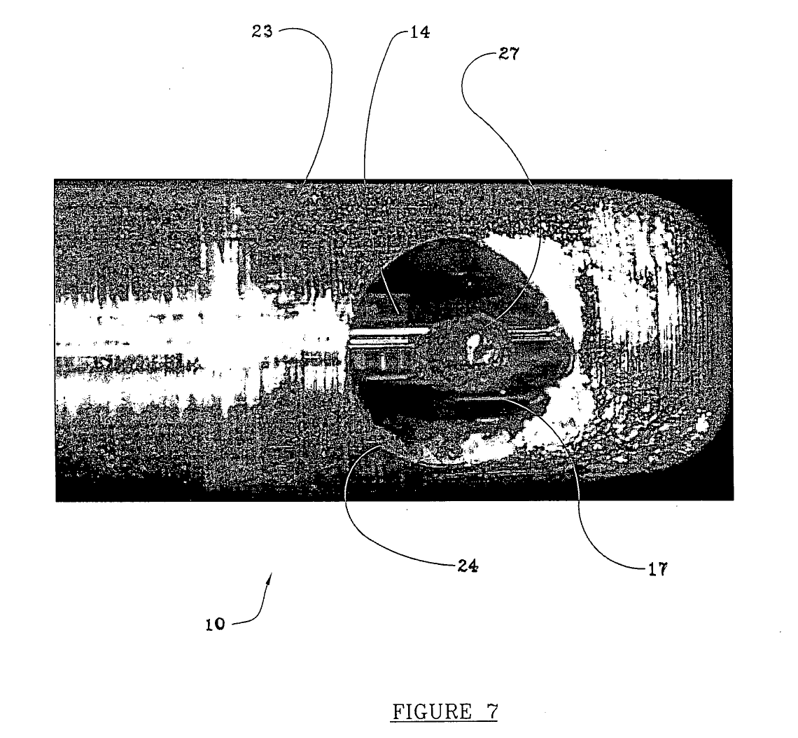

As seen in FIG. 7, in an image taken after transmission of 107 watts of Holmium laser power for ninety (90) minutes through side firing device 10 of the present invention on tissue, reflective metal sheath 23 shows some pitting distal to port 24, due to damage from laser energy reflected back from the target tissue and back splatter degradation during use.

This demonstrates that sheath 23 assists in protecting capillary tube 17 during use, as the back reflected laser energy, back splatter degradation and hydrothermal erosion, in the absence of reflective metal sheath 23, would have been incident on the distal end of capillary tube 17. Also, capillary tube 17 has been somewhat eroded due to hydrothermal erosion at area of laser energy emission 27, but has not been significantly damaged by laser energy reflected from laser energy reflected back from the target tissue and back splatter degradation during use. Also, the laser energy transmission efficiency of device 10 of the present invention has not been impaired to the point of unusability, it is not close to failure and laser energy can continue to be emitted from device 10.

A side firing device constructed in accordance with the preferred embodiments of the present invention described above can be used, for example, without limitation, for at least one of the following medical purposes:

(a) to vaporize excess, benign prostate tissue to relieve its obstructing urine flow (BPH);
(b) to vaporize excess nucleus pulposa tissue of a herniated spinal disc to relieve the pressure of the disc on nerves in the spine (laser disc decompression);
(c) to vaporize cartilaginous tissue of the facet and, if necessary, a small amount of non-load bearing bone of the facet, to enable an endoscope and the side firing device to gain entry to the foraminal space in the spine and vaporize nucleus pulposa tissue extruded from a ruptured spinal disc to relieve its pressure on nerves in the spine (endoscopic laser foraminoplasty);
(d) to coagulate/vaporize a uterine fibroid tumor, which bleeds profusely if cut, the side firing device is inserted and the tumor is coagulated/vaporized from the inside, by rotating the laser beam from the side firing device like the beacon of a lighthouse; and
(e) to coagulate/vaporize a solid tumor, the side firing device is inserted into the tumor, and the tumor is coagulated/vaporized from the inside, by rotating the laser energy beam from the side firing device like the beacon of a lighthouse.

While this invention is susceptible of embodiment in many different forms, there are shown in the drawings and will be described in detail herein specific embodiments thereof, with the understanding that the present disclosure is to be considered as an exemplification of the principles of the invention and is not to be limited to the specific embodiment illustrated.

Numerous variations and modifications of the embodiments described above can be effected without departing from the spirit and scope of the novel features of the invention. It is to be understood that no limitation with respect to the specific apparatus illustrated herein is intended or should be inferred. It is, of course, intended to cover by the appended claims, all such modifications as fall within the scope of the claims.

We claim:

1. A side firing laser device comprising
a conduit having an open distal end;
a closed end, transparent capillary tube defining a cavity and mounted to a distal end portion of the conduit so that the cavity is in communication with the conduit;
an optical fiber in the conduit, adapted for coupling to a laser energy source, including a proximal end portion for coupling to the laser energy source and a beveled distal end portion extending into said cavity;
a bulbous, internally reflective metal sheath about the capillary tube and mounted to the conduit;
the distal end portion of the optical fiber extending freely into said cavity; and
the metal sheath defining a side port providing an outwardly path for laser radiation emitted from the distal end of the optical fiber and an aperture aligned with optical axis of the optical fiber.

2. The side firing laser device in accordance with claim 1 wherein end surface of the beveled distal end portion defines an angle in the range of about 40 degrees to about 41 degrees with the optical axis of the optical fiber.

3. The side firing laser device in accordance with claim 1 wherein clearance between cavity sidewall and the beveled distal end portion is not more than 40 microns.

4. The side firing laser device in accordance with claim 1 wherein clearance between cavity sidewall and the beveled distal end portion is in the range of about 1 to 25 microns.

5. The side firing laser device in accordance with claim 1 wherein the beveled distal end portion terminates in a flat surface having a surface curvature of no more than about 5 microns.

6. The side firing laser device in accordance with claim 1 wherein the beveled distal end portion terminates in a flat surface having a surface curvature of no more than about 1.3 microns.

7. The side firing laser device in accordance with claim 1 wherein the optical fiber has a core diameter of about 450 microns and a core-to-cladding ratio of about 1:1.05.

8. The side firing laser device in accordance with claim 1 wherein the capillary tube has a wall thickness in the range of about 100 to about 1000 microns.

9. A side firing optical fiber device, comprised of a source of laser energy, an optical fiber optically coupled to the source of laser energy, the optical fiber having a core of fused silica and an exterior cladding of fused silica doped with a material that reduces its refractive index, the distal end of the optical fiber being beveled, and the beveled, distal end of the optical fiber being encased by a distally closed-ended, fused silica capillary tube, providing an air interface opposite the beveled, distal end surface of the optical fiber necessary for total internal reflection of laser energy, which includes at least one of the following:
(a) an optical fiber having an optimal core diameter of about 450 microns, able to efficiently transmit the high levels of laser energy with wavelengths commonly used through side firing devices;
(b) an optical fiber optimally having a cladding of fluorinated fused silica, preferably fluorinated synthetic fused silica, with a wall thickness of about 11 microns, for a core to cladding ratio of about 1:1.05, providing the necessary lower refractive index required for efficient transmission of laser energy through the optical fiber at substantially lower cost than the greater 1:1.1 to 1:1.2 core to cladding ratios commonly used in high power optical fibers;
(c) an optical fiber optimally having an outer overjacket of undoped fused silica, preferably undoped synthetic fused silica, with a wall thickness of about 65 microns, with a combined core to cladding and overjacket ratio of about 1:1.34, providing optimal protection to the thin, leading edge of the beveled, distal end surface of the optical fiber;
(d) an optical fiber having an optimal OD of about 600 microns, which is sufficiently small to enable as large as possible wall thickness of a distally closed-ended capillary tube to be used to encase the beveled, distal end portion of the bared optical fiber;
(e) an optical fiber having its distal end beveled at an angle of 35° to 45°, optimally at an angle of about 40° to 41° providing total internal reflection of laser energy at an angle of 80° to 82° laterally from the axis of the optical fiber;
(f) a beveled, distal end surface of the optical fiber having an optimal flatness, with a curvature across the surface of not more than about 5 microns, most optimally not more than 1.3 microns to minimize laser energy transmission losses;
(g) an optical fiber having taken a set while stored on a spool and, when unwound from the spool for further manufacture, using this set to optimally position the thin leading edge of the beveled, distal end surface of the optical fiber away from contact with the inner surface of the distally closed-ended capillary tube close-fitted over the beveled distal end portion of the optical fiber;
(h) a capillary tube having a wall thickness of about 100 to 1000 microns, most optimally about 510 microns, to maximize the device's functional longevity by increasing the capillary tube's resistance to damage and minimizing its susceptibility to hydrothermal erosion;
(i) a capillary tube being composed of fused silica, preferably fluorinated fused silica, most preferably fluorinated synthetic fused silica, to optimally increase the resistance of the capillary tube against damage due to back reflection of laser energy, back splatter degradation and hydrothermal erosion;
(j) a capillary tube optimally having an eccentric channel, the greater wall thickness of the capillary tube optimally being positioned at 180° opposite the beveled, distal end surface of the optical fiber, providing additional protection against damage to the capillary tube from back reflection of laser energy, back splatter degradation and hydrothermal erosion;
(k) a distal end of the capillary tube, after having been closed by thermal fusion, being annealed by reducing the temperature of the capillary tube in a series of timed steps to optimally reduce the presence of stresses in the capillary tube;
(l) a distal end of the capillary tube, after having been closed by thermal fusion and annealed, being further tempered by rapidly reducing the temperature of the outer surface of the capillary tube to optimally increase the hardness of the outer surface of the capillary tube;
(m) a capillary tube optimally being close-fitted and not fixedly attached over the distal end portion of the optical fiber, with a gap not exceeding 40 microns, creating a passageway for gasses trapped between the capillary tube and the distal end portion of the optical fiber to expand, when heated by the emission of laser energy, without creating excessive pressure on the capillary tube and the distal end portion of the optical fiber;
(n) a capillary tube being optimally fixedly attached and close-fitted within an outer, hollow, protective metal sheath coated with, or preferably composed of, a material highly reflective to the wavelengths of laser energy commonly used through side firing optical fiber devices, preferably a highly pure reflective metal, most preferably silver with a purity of about 99.5%, with a gap not exceeding 40 microns between the exterior of the capillary tube and the inner surface of the metal sheath, enabling the inner surface of the reflective metal sheath to reflect aberrant beams of laser energy from imperfections in the beveled, distal end surface of the optical fiber and the interior surface of the capillary tube, back through the capillary tube and out of the device, and enabling the outer surface of the reflective metal sheath to protect the capillary tube from back reflected laser energy, back splatter degradation and hydrothermal erosion;
(o) a hollow metal sheath having a port for emission of laser energy positioned at 180° opposite the beveled, distal end surface of the optical fiber and a distal end opening to optimally allow forwardly emitted laser energy to escape, without overheating the distal end of the metal sheath and the capillary tube;
(p) a hollow metal sheath having a wall thickness of about 300 microns, optimally bringing the overall outside diameter of the side firing optical fiber device no more than about 2.3 mm, enabling the side firing device to optimally pass into, move within and be withdrawn from the instrument channel of commonly available medical endoscopes;
(q) a rigid, hollow shaft composed of one of: metal or plastic, preferably composed of medical grade stainless steel, with a wall thickness of about 300 microns, disposed over the optical fiber and extending from about the middle of the length of the capillary tube to about the proximal end of a handpiece provided for ease of use by the operator, enabling the side firing optical fiber device to optimally resist rapid movement and vibration due to the equal and opposite forces exerted against the side firing device by the emission of laser energy, and enabling the side firing device to more easily being kept in place opposite the target tissue by the operator.
(r) an optical fiber being fixedly attached to the interior of the rigid, hollow shaft near the proximal end of the hollow shaft, to optimally allow movement of the beveled, distal end portion of the optical fiber within the capillary tube during handling, insertion into, during use and withdrawal of the side firing device from the working channel of an endoscope and optimally avoiding stress on the optical fiber which could lead to premature failure of the device;
(s) a rigid, hollow shaft disposed over the optical fiber creating a channel enabling gasses trapped between the capillary tube and the beveled, distal end portion of the optical fiber to optimally expand, when heated during the emission of laser energy, preventing excessive pressure that can damage the capillary tube and the optical fiber;
(t) a rigid, hollow metal shaft having at least one vent near its proximal end, distal to the point at which the optical fiber is fixedly attached to the interior of the hollow rigid shaft, to allow gasses trapped between the capillary tube and the beveled, distal end surface of the optical fiber, when heated by the emission of laser energy, to optimally escape into the atmosphere, without creating excessive pressure against the capillary tube and the distal end portion of the optical fiber;
(u) a durable, lubricious outer sleeve of plastic, preferably composed of PEEK, with a wall thickness of about 125 microns, fixedly attached to the metal shaft, enabling the side firing device to optimally pass into, be used within and be withdrawn from the instrument channel of commonly available endoscopes without scuffing and without excessive forces;
(v) a durable, lubricious outer coating, with a wall thickness of at least 0.1 microns, fixedly attached to the hollow shaft, enabling the side firing device to optimally pass into, be used within and be withdrawn from the instrument channel of commonly available endoscopes without scuffing and without excessive forces; and
(w) a high temperature optically transparent epoxy, adhesive allowing the device to withstand the elevated peak and average temperatures created at the end of the side firing device during use, and which does not significantly absorb the wavelength of laser energy being used.

10. The side firing optical fiber device of claim 9, wherein the source of laser energy emits at a wavelength of at one of: 300 to 400 nm, 400 to 1400 nm, 1500 to 1800 nm, 1400 to 1500 nm, 1800 to 2300 nm.

11. The side firing optical fiber device of claim 9, wherein the source of laser energy is one of: an excimer laser emitting at a wavelength of one of: 308 and 351 nm, a KTP laser emitting at a wavelength of 532 nm, a diode laser emitting at a wavelength from 635 to 1100 nm, a diode laser emitting at a wavelength of about 1470 nm, a diode laser emitting at a wavelength of about 1940 nm, a Thulium:YAG laser emitting at a wavelength of 2000 nm and a CTH:YAG laser emitting at a wavelength of 2100 nm.

12. A side firing optical fiber device, comprised of a source of laser energy, an optical fiber optically coupled to the source of laser energy, the optical fiber having a core of fused silica and an exterior cladding of fused silica doped with fluorine in an amount sufficient to reduce its refractive index, the distal end of the optical fiber being beveled, and the beveled, distal end of the optical fiber being encased by a distally closed-ended, fused silica capillary tube, providing an air interface opposite the beveled, distal end surface of the optical fiber necessary for total internal reflection of laser energy, and including at least one of the following:
(a) an optical fiber having an optimal core diameter of about 450 microns, able to efficiently transmit the high levels of laser energy with wavelengths commonly used through side firing devices;
(b) an optical fiber optimally having a cladding of fluorinated, fused silica with a wall thickness of about 11 microns, with core-to-cladding ratio of about 1:1.05.
(c) an optical fiber optimally having an outer overjacket of undoped fused silica, preferably undoped synthetic fused silica, with a wall thickness of about 65 microns, with a combined core to cladding and overjacket ratio of about 1:1.34, providing optimal protection to the thin, leading edge of the beveled, distal end surface of the optical fiber;
(d) an optical fiber having its distal end beveled at an angle of 35° to 45°, optimally at an angle of about 40° to 41° providing optimal total internal reflection of laser energy at an angle of 80° to 82° laterally from the axis of the optical fiber;
(e) a beveled, distal end surface of the optical fiber having an optimal flatness, with a curvature across the surface of not more than about 5 microns, most optimally not more than 1.3 microns to minimize laser energy transmission losses;
(f) an optical fiber having taken a set while stored on a spool and, when unwound from the spool for further manufacture, using this set to optimally position the thin leading edge of the beveled, distal end surface of the optical fiber away from contact with the inner surface of the distally closed-ended capillary tube enclosing the beveled distal end portion of the optical fiber;
(g) a capillary tube being composed of fused silica, preferably fluorinated fused silica, most preferably fluorinated synthetic fused silica, to optimally increase the resistance of the capillary tube against damage due to back reflection of laser energy, back splatter degradation and hydrothermal erosion;
(h) a distal end of the capillary tube, after having been closed by thermal fusion, being annealed by reducing the temperature of the capillary tube in a series of timed steps to optimally reduce the presence of stresses in the capillary tube;
(i) a distal end of the capillary tube, after having been closed by thermal fusion and annealed, being further tempered by rapidly reducing the temperature of the outer surface of the capillary tube to optimally increase the hardness of the outer surface of the capillary tube; and
(j) a capillary tube being optimally fixedly attached and close-fitted within an outer, hollow, protective metal sheath composed of a material highly reflective to the wavelengths of laser energy commonly used through side firing optical fiber devices, with a gap not exceeding 40 microns, enabling the inner surface of the reflective metal sheath to reflect aberrant beams of laser energy back through the capillary tube and out of the device.

13. A side firing optical fiber device, comprised of a source of laser energy, an optical fiber optically coupled to the source of laser energy, the optical fiber having a core of fused silica and an exterior cladding of fused silica doped with a material, such as fluorine, to reduce its refractive index, the distal end of the optical fiber being beveled at an angle of 35° to 45°, and the beveled, distal end of the optical fiber being encased by a distally closed-ended, fused silica capillary tube, providing an air interface opposite the beveled, distal end surface of the optical fiber necessary for total internal reflection of laser energy, and including at least one of the following:
(a) an optical fiber having an optimal OD of about 600 microns, which is sufficiently small to enable as large as possible wall thickness of a distally closed-ended capillary tube to be used to encase the beveled, distal end portion of the bared optical fiber;
(b) a capillary tube having a wall thickness of about 100 to 1000 microns, most optimally about 510 microns, to maximize the device's functional longevity by increasing the capillary tube's resistance to damage and minimizing its susceptibility to hydrothermal erosion;
(c) a capillary tube being composed of fused silica, preferably fluorinated fused silica, most preferably fluorinated synthetic fused silica, to optimally increase the resistance of the capillary tube against damage due to back reflection of laser energy, back splatter degradation and hydrothermal erosion;
(d) a capillary tube optimally having an eccentric channel, the greater wall thickness of the capillary tube optimally being positioned at 180° opposite the beveled, distal end surface of the optical fiber, providing additional protection against damage to the capillary tube from back reflection of laser energy, back splatter degradation and hydrothermal erosion;
(e) a distal end of the capillary tube, after having been closed by thermal fusion, being annealed by reducing the temperature of the capillary tube in a series of timed steps to optimally reduce the presence of stresses in the capillary tube;
(f) a distal end of the capillary tube, after having been closed by thermal fusion and annealed, being further tempered by rapidly reducing the temperature of the outer surface of the capillary tube to optimally increase the hardness of the outer surface of the capillary tube;
(g) a capillary tube optimally being close-fitted and not fixedly attached over the distal end portion of the optical fiber, with a gap not exceeding 40 microns, creating a passageway for gasses trapped between the capillary tube and the distal end portion of the optical fiber to expand, when heated by the emission of laser energy, without creating excessive pressure on the capillary tube and the distal end portion of the optical fiber;
(h) a hollow sheath disposed over the capillary tube, being optimally composed of a material highly reflective to the wavelengths of laser energy commonly used through side firing optical fiber devices, preferably a highly pure reflective metal, most preferably silver with a purity of about 99.5%, to provide optimal protection to the capillary tube against damage from back reflected laser energy, back splatter degradation and hydrothermal erosion;
(i) a hollow sheath having a port for emission of laser energy optimally positioned at 180° opposite the beveled, distal end surface of the optical fiber and a distal open end to optimally allow forwardly emitted laser energy to escape, without overheating the distal end of the metal sheath and the capillary tube;
(j) a rigid, hollow shaft comprised of one of: metal or plastic, preferably composed of medical grade stainless steel, with a wall thickness of about 300 microns, disposed over the optical fiber and extending from about the middle of the length of the capillary tube to about the proximal end of a handpiece provided for ease of use by the operator, enabling the side firing optical fiber device to optimally resist rapid movement and vibration due to the equal and opposite forces exerted against the side firing device by the emission of laser energy, and enabling the side firing device to more easily being kept in place opposite the target tissue by the operator;
(k) an optical fiber being fixedly attached to the rigid hollow shaft near the proximal end of the hollow shaft, to optimally allow movement of the optical fiber within the capillary tube during handling, insertion into, during use and withdrawal of the side firing device from the working channel of an endoscope and optimally reducing stresses on the optical fiber; and
(l) a rigid shaft disposed over the optical fiber creating a channel enabling gasses trapped between the capillary tube and the beveled, distal end portion of the optical fiber to optimally expand, when heated during the emission of laser energy, and the rigid shaft having at least one vent near its proximal end to enable such gasses to escape into the atmosphere, preventing excessive pressure that can damage the capillary tube and the optical fiber.

14. A side firing optical fiber device, comprised of a source of laser energy, an optical fiber optically coupled to the source of laser energy, the optical fiber having a core of fused silica and an exterior cladding of fused silica doped with as fluorine, in an amount sufficient to reduce its refractive index, the distal end of the optical fiber being beveled at an angle of 35° to 42°, and the beveled, distal end of the optical fiber being encased by a distally closed-ended, fused silica capillary tube, providing an air interface opposite the beveled, distal end surface of the optical fiber necessary for total internal reflection of laser energy, comprised of at least one of:
(a) a hollow shaft comprised of one of: metal or plastic, disposed over the optical fiber, extending from about the middle of the length of the capillary tube to about the proximal end of a handpiece provided for ease of use by the operator, the hollow shaft having a wall thickness of about 300 microns, enabling the side firing optical fiber device to optimally resist rapid vibration from the equal and opposite forces exerted on the side firing device from the emission of laser energy, and enabling the side firing optical fiber device to be more easily kept in position opposite the target tissue by the operator;
(b) a durable, lubricious outer sleeve of plastic, preferably composed of PEEK, with a wall thickness of about 125 microns, fixedly attached to the hollow shaft, enabling the side firing device to optimally pass into, be used within and be withdrawn from the instrument channel of commonly available endoscopes without scuffing and without excessive forces; and
(c) a durable, lubricious outer coating, with a wall thickness of at least 0.1 microns, fixedly attached to the hollow shaft, enabling the side firing device to optimally pass into, be used within and be withdrawn from the instrument channel of commonly available endoscopes without scuffing and without excessive forces.

15. A side firing optical fiber device capable of a consistently high rate of vaporization of tissue, extended longevity, high reliability and improved handling, comprised of a source of laser energy, an optical fiber optically coupled to the source of laser energy, the optical fiber having a core of fused silica with a diameter of about 450 microns and a cladding of fluorine doped fused silica, preferably fluorine doped synthetic fused silica, with a wall thickness of about 11 microns, with an overjacket of undoped fused silica, preferably undoped synthetic fused silica, with a wall thickness of about 65 microns, the distal end of the optical fiber being beveled at an angle of 40° to 41°, and the distal end portion of the optical fiber being disposed within a distally closed-ended capillary tube of fused silica, preferably of synthetic fused silica and most preferably of fluorine doped synthetic fused silica, with a wall thickness of about 510 microns, providing an air interface opposite the beveled, distal end surface of the optical fiber necessary for total internal reflection of laser energy.

16. The side firing optical fiber device of claim 15, wherein to increase its functional longevity and reliability, a reflective metal sheath is disposed over the capillary tube to protect the capillary tube from damage during use.

17. The side firing optical fiber device of claim 15, wherein to eliminate the glass to air to glass interfaces that reduces the overall laser energy transmission efficiency by about 7%, the capillary tube is thermally fused to the optical fiber at one of: opposite the beveled, distal end surface of the optical fiber or along the line 180° opposite the bevel of the distal end surface of the optical fiber, and further processed by one of: during the fusing process or after the fusing process, annealing the capillary tube and bared, distal end portion of the optical fiber by a series of timed, small reductions in temperature, while disposing a portion of the optical fiber proximal to the capillary tube in an enclosure through which a cooled fluid is circulated, to optimally reduce the presence of stresses in the capillary tube and the optical fiber.

18. The side firing optical fiber device of claim 15, wherein to resist vibration from the equal and opposite forces exerted on the side firing device, the optical fiber is enclosed within a rigid, hollow shaft comprised of one of: metal or plastic, preferably of medical grade stainless steel with a wall thickness of about 300 microns, which shaft extends from about the middle of the length of the capillary tube to about the proximal end of a handpiece provided for ease of use by the operator.

19. The side firing optical fiber device of claim 15, wherein the capillary tube defines an eccentric channel providing a greater wall thickness of the capillary tube to be optimally disposed 180° opposite the beveled, distal end surface of the optical fiber.
